(19) United States
(12) Patent Application Publication
(10) Pub. No.: US 2007/0213368 A1
(43) Pub. Date: Sep. 13, 2007

(54) N-ETHYL HYDROXYETHYLAMINE USEFUL IN TREATING CNS CONDITIONS

(76) Inventors: Edward F. Kleinman, Pawcatuck, CT (US); John C. Murray, East Lyme, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/575,740

(22) PCT Filed: Sep. 9, 2005
(86) PCT No.: PCT/IB05/02878

§ 371 Date: Mar. 21, 2007

Related U.S. Application Data
(60) Provisional application No. 60/611,685, filed on Sep. 21, 2004.

(51) Int. Cl. A61K 31/455; A61K 31/4172; A61K 31/405; A61K 31/277; A61K 31/18; A61K 31/165

(52) U.S. Cl. 514/315; 514/400; 514/419; 514/617; 514/563; 514/602; 514/616; 546/315; 548/335.5; 548/495; 562/450; 564/86; 564/152; 564/174; 514/521; 558/410

(57) ABSTRACT

An N-ethyl hydroxyethyleneamine of formula (I) are disclosed which are useful in treating CNS conditions, including neurodegenerative diseases such as Alzheimer's Disease.

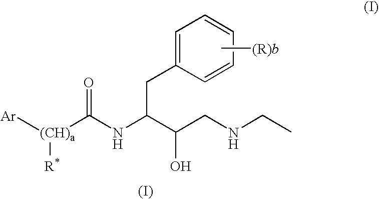

The invention pertains to an N-ethyl hydroxyethylamine compound useful e.g. in treating conditions of the Central Nervous System (CNS); a pharmaceutical composition comprising same; and a method of treating such conditions and those in which inhibition of beta-secretase is indicated.

### BACKGROUND OF THE INVENTION

Conditions affecting the Central Nervous System include neurodegenerative conditions such as Alzheimer's Disease. Various of these conditions are typified by physical changes in the brain. For example, certain pathologies are evidenced by the presence of neurofibrillary tangles and/or plaque deposits which, as they progress, cause cognitive, motor, sensory and other impairments on multiple fronts. Commonly, said plaques are comprised principally of beta-amyloid—a highly aggregative protein that tends to accumulate, forming insoluble deposits that ultimately can cause cellular injury and death. Beta-amyloid (Aβ) derives from an amyloid precursor protein (APP), which is a transmembrane protein existing in several isoforms, the more salient of which contain 695, 714, 751 or 771 amino acids (denominated APP₆₉₅, APP₇₁₄, APP₇₅₁, APP₇₇₁). The formation of beta-amyloid is due to the sequential cleavage of APP by various proteases: beta-secretease cleaves APP at an N-terminus while gamma-secretase cleaves APP at a C-terminus. The resulting fragment is a protein of 38, 40, 42 or 43 amino acids (denominated Aβ₁₋₃₈, Aβ₁₋₄₀, Aβ₁₋₄₂, Aβ₁₋₄₃). This fragment is released into the extracellular space where it accumulates with other such insoluble fragments to form the proteinacious deposits aforesaid that are neuronally toxic.

Among the treatment strategies under investigation for such conditions are the development of compounds that will effectively inhibit beta-secretase and/or its processing of APP to reduce the formation of beta-amyloid and ameliorate plaque deposition and related pathogenesis.

### SUMMARY OF THE INVENTION

The present invention is directed to an N-ethyl hydroxyethylamine compound of Formula (I) having beta-secretase inhibitory characteristics:

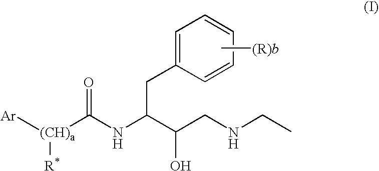

### DETAILED DESCRIPTION OF THE INVENTION

The compound of the invention as represented by the above formula includes all stereoisomieric forms including without limitation the (R) or (S) enantiomer thereof, diastereomers, or a pharmaceutically acceptable salt, solvate or prodrug thereof, or of any of the foregoing. Pharmaceutically acceptable salts include acid addition salts, base addition salts and the like as understood by and as fabricated according to methods known in the art. The present compound may also have optical centers and thus occur in different enantiomeric configurations, all of which are contemplated herein. The compound of the invention further includes radiolabelled forms wherein e.g. one or more H, C, F atoms and the like are replaced with radioactive species of the same.

As appreciated by the artisan, the use of Formula I is a convenience and the invention is understood to envision and embrace each and every species thereunder as though individually identified and set forth herein. Thus the present invention severally contemplates each species separately and any and all combinations and permutations of species falling within Formula I.

Turning to Formula (I): in one embodiment a=0, 1, 2, or 3; b=0, 1, 2, or 3; each R is independently halogen, OH, CN, SH, NH₂, C₁₋₆alkyl, C₁₋₆alkoxy, S(C₁₋₆alkyl), NH(C₁₋₆alkyl), N(C₁₋₆alkyl)(C₁₋₆alkyl), NHC(═O)O(C₁₋₆alkyl), NHSO₂(C₁₋₆alkyl), C(═O)NH(C₁₋₆alkyl), C(═O)N(C₁₋₆alkyl)(C₁₋₆alkyl), C₆₋₁₀aryl, (5 to 12 member) heteroaryl, wherein each alkyl group aforesaid may be independently optionally substituted with up to three F, OH or C₁₋₃alkoxy groups. As noted, each R may independently be chosen from the foregoing, i.e. each and every R can be the same or different irrespective of the value of b; R* is H, C₁₋₆alkyl, —(CH₂)₀₋₅(C₆-C₁₀aryl), —(CH₂)₀₋₅ (5 to 12 member) heteroaryl; and Ar is selected from (A), (B), (C), (D), (E) or (F):

(A) C₆₋₁₀aryl, (5 to 12 member) heteroaryl, (C₆₋₁₀aryl)-W—(C₆₋₁₀aryl), (C₆₋₁₀aryl)-W-(5 to 12 member) heteroaryl, (C₆₋₁₀aryl)-W-(5 to 7 member) heterocycloalkyl, (5 to 12 member) heteroaryl-W—(C₆₋₁₀aryl), (5 to 12 member) heteroaryl-W-(5 to 12 member) heteroaryl, (5 to 12 member) heteroaryl-W-(5 to 7 member) heterocycloalkyl, (5 to 7 member) heterocycloalkyl-W—(C₆₋₁₀aryl), (5 to 7 member) heterocycloalkyl-W-(5 to 12 member) heteroaryl, (5 to 7 member) heterocycloalkyl-W-(5 to 7 member) heterocycloalkyl, wherein W is selected from —(CH₂)₀₋₄—, —O—, —C(═O)—, —S(═O)₀₋₂—, —N(R_{N-5})— where R_{N-5} is as defined herein;

(B) —C(═O)(C₁₋₁₀alkyl) where alkyl is optionally independently substituted with up to three substitutents (denominated herein as “SB”) selected from: OH; C₁₋₆alkoxy; C₁₋₆thioalkoxy; C(═O)OR_{N-8}; —C(═O)NR_{N-2}R_{N-3}; —C(═O)R_{N-4}; —SO₂(C₁₋₈alkyl); —OS₂NR_{N-2}R_{N-3}; —NHC(═O)(C₁₋₆alkyl); —NHC(═O)OR_{N-6}; —NR_{N-2}R_{N-3}; —R_{N-4}; —OC(═O)(C₁₋₆alkyl); —O—C(═O)NR_{N-8}R_{N-8} where each R_{N-8} is the same or different; —O(C₁₋₆alkyl)C(═O)OH; —O—(C₁₋₆alkyl optionally substituted with up to three halogens); —NHSO₂(C₁₋₆ alkyl); F; Cl;

(C) —C(═O)(C₁₋₆alkyl)O(C₁₋₆alkyl) where each alkyl is optionally independently substituted with up to three substituents SB as defined above in (A);

(D) —C(═O)(C₁₋₆alkyl)S(C₁₋₆alkyl) where each alkyl is optionally independently substituted with up to three of substituents SB as defined above in (A);

(E) —C(═O)CH(—(CH₂)₀₋₂—O—R_{N-10})—(CH₂)₀₋₂—C₆₋₁₀aryl, or —C(═O)CH(—(CH₂)₀₋₂—O—R_{N-10})—(CH₂)₀₋₂— (5 to 12 member) heteroaryl; or

(F) —C(═O)(C₃₋₈cycloalkyl) where said -cycloalkyl is optionally independently substituted with up to two substituents selected from: —(CH₂)₀₋₄OH; —(CH₂)₀₋₄C₁₋₆alkoxy; —(CH₂)₀₋₄C₁₋₆thioalkoxy; —(CH₂)₀₋₄C(═O)—O—R_{N-8}; —(CH₂)₀₋₄C(═O)—NR_{N-2}R_{N-3}; —(CH₂)₀₋₄C(═O)—R_{N-4}; —(CH₂)₀₋₄SO₂—(C₁₋₆alkyl); —(CH₂)₀₋₄SO₂—NR_{N-2}R_{N-3}; —(CH₂)₀₋₄NH—C(═O)—(C₁₋₆alkyl); —NH—C(═O)—O—R_{N-8}; —(CH₂)₀₋₄NR_{N-2}R_{N-3}; —(CH₂)₀₋₄R_{N-4}; —O—C(═O)—(C₁₋₆alkyl); —O—C(═O)—NR_{N-8}R_{N-8} where each R_{N-8} is the same or different; —O—(C₁₋₆alkyl)—C(═O)OH; —O—(C₁₋₆alkyl, wherein said alkyl is optionally substituted with up to three halogens); —NHSO₂(C₁₋₆alkyl); F; Cl.

Unless otherwise indicated, the following representative definitions of terms and substituents and related variations of same obtain:

“Halogen” and “halo” and the like independently includes fluoro (F), chloro (Cl), bromo (Br) and iodo (I).

“Alkyl” including as may appear in the terms “alkoxy,” “thioalkoxy” and “alkyoxy” and the like includes saturated monovalent hydrocarbon radicals having straight or branched moieties. Examples of alkyl groups include, but are not limited to, methyl, ethyl, n-propyl, isopropyl, and and t-butyl.

“Alkenyl” and “Alkynyl” include alkyl moieties having at least one carbon-carbon double or triple bond, respectively.

“Cycloalkyl” includes non-aromatic saturated cyclic alkyl moieties wherein alkyl is defined as above. Examples included without limitation: cyclopropyl, cyclobutyl, cyclopentyl, cyclohexyl, cycloheptyl; and bicycloalkyl and tricycloalkyl groups that are non-aromatic saturated carbocyclic groups consisting of two or three rings respectively wherein said rings share at least one carbon atom. Unless otherwise indicated herein bicycloalkyl groups include spiro groups and fused ring groups, e.g. bicycle-[3.1.0]-hexyl, bicycle-[2.2.1]-hept-1-yl, norbornyl, spiro[4.5]decyl, spiro[4.4]nonyl, spiro[4.3]octyl and spiro[4.2]heptyl. An example of a tricycloalkyl group is adamantanyl. Cycloalkyl groups also include groups substituted with one or more oxo moieties, e.g. oxocyclopentyl and oxocyclobutyl.

As appreciated, the term (CH₂)₀₋₅ and the like denotes the -optional presence of a methylene linkage up to the carbon number indicated (here, 5), the connecting substituent,to which may be in the normal or branched configuration, e.g. for (CH₂)₀₋₅(C₆₋₁₀aryl) the aryl may be in the branched or normal position in the methylene chain.

The terms “alkyl,” “alkoxy,” “thioalkoxy,” “alkyoxy,” “alkenyl,” “alkynyl,”“cycloalkyl” as defined and used herein are further intended to include moieties of same that may each be optionally substituted with up to 3 fluoro atoms (F) irrespective of whether such substitutions are specifically mentioned herein as optional or otherwise.

“Treatment” and “treating” refers to reversing, alleviating, inhibiting the progress of, or preventing the disorder or condition to which such term applies, or one or more symptoms of such condition or disorder. As used herein, the term also encompasses, depending on the condition of the patient, preventing the disorder, including preventing onset and/or recurrence of any symptoms associated therewith, as well as reducing the severity of the disorder or any of its symptoms prior to onset.

“Mammal” refers to any member of the class “Mammalia”, including, but not limited to, humans, dogs, and cats.

“Condition” refers to a disease or disorder.

“Aryl” refers to an organic radical derived from an aromatic hydrocarbon by removal of one hydrogen; and fused ring groups wherein at least one ring is aromatic. Examples without limitation include: phenyl, 1-naphthyl, 2-naphthyl, tetralinyl, indanyl, dihydronaphthyl, indenyl, fluorenyl and 6,7,8,9-tetrahydro-5H-benzo[a]cycloheptenyl. Aryl groups contemplated herein may further be optionally independently substituted with up to three of any of the following substituents (1)-(39): (1) —C₁₋₆alkyl, optionally substituted with up to three substituents selected from C₁₋₃alkyl, halogen, OH, SH, CN, CF₃, C₁₋₃alkoxy, NR₁₋ₐR_{1-b} where R₁₋ₐ and R_{1-b}; such C₁₋₆ alkyl-substituted aryl groups include, e.g. benzyl; (2) OH; (3) NO₂; (4) halogen, with F being preferred; (5) —C(═O)OH; (6) —CN; (7) —(CH₂)₀₋₄C(═O)NR_{N-2}R_{N-3}; (8) —(CH₂)₀₋₄C(═O)(C₁₋₂alkyl); (9) —(CH₂)₀₋₄C(═O)(C₂₋₁₂alkenyl with bonds); (10) —(CH₂)₀₋₄C(═O)(C₂₋₁₂alkynyl with one, two or three triple bonds);. (11) —(CH₂)₀₋₄C(═O)(C₃₋₇cycloalkyl); (12) —(CH₂)₀₋₄C(═O)(C₆₋₁₀aryl); (13) —(CH₂)₀₋₄C(═O) (5 to 12 member) heteroaryl; (14) —(CH₂)₀₋₄C(═O) (5 to 7 member) heterocycloalkyl; (15) —(CH₂)₀₋₄C(═O)R_{N-4}; (16) —(CH₂)₀₋₄C(═O)OR_{N-5}; (17) —(CH₂)₀₋₄SO₂—NR_{N-2}R_{N-3}; (18) —(CH₂)₀₋₄S(═O)(C₁₋₆alkyl); (19) —(CH₂)₀₋₄SO₂—(C₁₋₁₂alkyl); (20) —(CH₂)₀₋₄SO₂(C₃₋₇cycloalkyl); (21) —(CH₂)₀₋₄N(H or R_{N-5})C(═O)OR_{N-5} where each R_{N-5} can be the same or different; (22) —(CH₂)₀₋₄N(H or R_{N-5})—C(═O)N(R_{N-5})₂, where each R_{N-5} can be the same or different; (23) —(CH₂)0-4N—C(═S)N(R_{N-5})₂, where each R_{N-5} can be the same or different; (24) —(CH₂)₀₋₄N(H or R_{N-5})—C(═O)R_{N-2}; (25) —(CH₂)₀₋₄NR_{N-2}R_{N-3}; (26) —(CH₂)₀₋₄R_{N-4}; (27) —(CH₂)₀₋₄OC(═O)(C₁₋₆alkyl); (28) —(CH₂)₀₋₄OP(═O)—(O—C₆₋₁₀aryl)₂; (29) —(CH₂)₀₋₄OC(═O)N(R_{N-5})₂ where each R_{N-5} can be the same or different; (30) —(CH₂)₀₋₄OC(═S)N(R_{N-5})₂ where each R_{N-5} can be the same or different; (31) —(CH₂)₀₋₄O(R_{N-5})₂ where each R_{N-5} can be the same or different; (32) —(CH₂)₀₋₄O(R_{N-5})₂—C(═)OH where each R_{N-5} can be the same or different; (33) —(CH₂)₀₋₄S(R_{N-5})₂ where each R_{N-5} can be the same or different; (34) —(CH₂)₀₋₄O(C₁₋₆alkyl optionally substituted with up to five F as obtains); (35) C₃₋₇cycloalkyl; (36) C₂₋₆alkenyl with one or two double bonds, said alkenyl optionally substituted with C₁₋₃alkyl, halogen, OH, SH, CN, CF₃, C₁₋₃alkoxy, NR₁₋ₐR_{1-b}; (37) —C₂₋₆alkynyl with one or two triple bonds, said alkynyl optionally substituted with C₁₋₃alkyl, halogen, OH, SH, CN, CF₃, C₁₋₃alkoxy, NR₁₋ₐR_{1-b}; (38) —(CH₂)₀₋₄N(H or R_{N-5})SO₂R_{N-2}; or (39) —(CH₂)₀₋₄C₃₋₇cycloalkyl.

“Heteroaryl” refers to a heteroaryl group constituted of one or more aromatic groups containing one or more heteroatoms (O, S, or N), preferably from one to four heteroatoms. As used herein, a multicyclic group containing one or more heteroatoms wherein at least one ring of the group is aromatic is also a “heteroaryl” group. The heteroaryl groups of this invention can also include ring systems which exist in one or more tautomeric forms (e.g. keto, enol, and like forms), and/or substituted with one or more oxo moieties. Examples of heteroaryl groups are, without limitation: quinolyl, isoquinolyl, 1,2,3,4-tetrahydroguinolyl, 1,2,4-trizainyl, 1,3,5-triazinyl, 1-oxoisoindolyl, furazanyl, benzofurazanyl, benzothiophenyl, dihydroquinolyl, dihydroisoquinolyl, benzofuryl, furopyridinyl, pyrolopyrimidinyl, and azaindolyl, pyridinyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Each heteroaryl may also be optionally independently substituted with up to four of any of the following substituents (1)-(13): (1) C₁₋₆alkyl, said alkyl optionally substituted with up to three substituents selected from C₁₋₃alkyl, halogen, OH, SH, NR₁₋ₐR_{1-b}, CN, CF₃, C₁₋₃alkoxy; (2) C₂₋₆alkenyl with one or two double bonds, said alkenyl optionally substituted with up to three substituents selected from F; Cl, OH, SH, CN, CF₃, C₁₋₃akoxy, NR₁₋ₐR_{1-b}; (3) C₂₋₆alkynyl with one or two triple bonds, said alkynyl optionally substituted with up to three substituents selected from F, Cl, OH, SH, CN, CF₃, C₁₋₃alkoxy, NR₁₋ₐR_{1-b}; (4) halogen; (5) C₁₋₆alkoxy, said alkoxy optionally substituted with up to F; (6) NR_{N-2}R_{N-3}; (7) OH; (8) CN; (9) C₃₋₇cycloalkyl, said cycloalkyl optionally substituted-with up to three substituents selected from F, Cl, OH, SH, CN, CF₃, C₁₋₃alkoxy, NR₁₋ₐR_{1-b}; (10) C(═(O)(C₁₋₄alkyl); (11) SO₂NR₁₋ₐR_{1-b}; (12) C(═O)NR₁₋ₐR_{1-b}; (13) SO₂(C₁₋₄alkyl).

“Heterocycloalkyl” and “Heterocyclic” refer to a heterocycloalkyl group of one or more non-aromatic cyclic groups containing one or more heteroatoms, preferably from one to four heteroatoms, each selected from O, S and N. Heterocyclic groups also include ring systems substituted with one or more oxo moieties. Without limitation, examples of heterocyclic groups include: aziridinyl, azetidinyl, azepinyl, 1,2,3,6-tetrahydropyridinyl, oxiranyl, oxetanyl, tetrahydrothiopyranyl, morpholino, thiomorpholino, indolinyl, 2H-pyranyl, 4H-pyranyl, dioxanyl, 1,3-dioxolanyl, pyrazolinyl, dihydrothienyl, dihydrofuranyl, pyrazolidinyl, imidazolinyl, imidazolidinyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl, morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide. Each heterocycloalkyl may also be optionally independently substituted with up to four of any of the following substituents (1)-(14): (1) C₁₋₆alkyl, said alkyl optionally substituted with up to three substituents selected from C₁₋₃alkyl, halogen, OH, SH, NR₁₋ₐR_{1-b}, CN, CF₃, C₁₋₃alkoxy; (2) C₂₋₆alkenyl with one or two double bonds, said alkenyl optionally substituted with up to three substituents selected from F, Cl, OH, SH, CN, CF₃, C₁₋₃alkoxy, NR₁₋ₐR_{1-b}; (3) C₂₋₆alkynyl with one or two triple bonds, said alkynyl optionally substituted with up to three substituents selected from F, Cl, OH, SH, CN, CF₃, C₁₋₃alkoxy, NR₁₋ₐNR_{1-b}; (4) halogen; (5) C₁₋₆alkoxy, said alkoxy optionally substituted with up to three F; (6) NR_{N-2}R_{N-3}; (7) OH; (8) CN; (9) C₃₋₇cycloalkyl, said cycloalkyl optionally substituted with up to three substituents selected from F, Cl, OH, SH, CN, CF₃, C₁₋₃alkoxy, NR₁₋ₐR_{1-b}; (10) C(═O)(C₁₋₄alkyl); (11) SO₂NR₁₋ₐR_{1-b}; (12) C(═O)NR₁₋ₐR_{1-b}; (13) —SO₂(C₁₋₄alkyl); (14) ═O.

The foregoing groups, as derived from the compounds listed above, may be C— attached or N-attached where such is possible. For instance, a group derived from pyrrole may be pyrrol-1-yl (N-attached) or pyrrol-3-yl (C-attached). The terms referring to the groups also encompass all possible tautomers.

“R₁₋ₐ” and “R_{1-b}” are each independently H, C₁₋₆ alkyl.

“R_{N-2}” and “R_{N-3}” are each independently selected from the group (a) H; (b) C₁₋₆alkyl optionally substituted with one substituent selected from: OH or NH₂; (c) C₁₋₆alkyl optionally substituted with up to three halogen; (d) C₃₋₇cycloalkyl; (e) —(C₁₋₂alkyl)(C₃₋₇cycloalkyl); (f) —(C₁₋₆alkyl)O(C₁₋₃alkyl); (g) C₂₋₆alkenyl with one or two double bonds; (h) C₂₋₆alkynyl with one or two triple bonds; (i) C₁₋₆alkyl chain with one double bond and one triple bond; (j) C₆₋₁₀aryl; or (k) (5 to 12 member) heteroaryl.

“R_{N-4}” is selected from the group: morpholinyl, thiomorpholinyl, piperazinyl, piperidinyl, homomorpholinyl, homothiomorpholinyl, homothiomorpholinyl S-oxide, homothiomorpholinyl S,S-dioxide, pyrrolinyl and pyrrolidinyl where each group is optionally substituted with one, two, three, or four of C₁₋₆alkyl.

“R_{N-5}” is selected from the group: (a) C₁₋₆alkyl, (b) —(CH₂)₀₋₂(C₆₋₁₀aryl), (c) C₂₋₆alkenyl containing one or two double bonds, (d) C₂₋₆alkynyl containing one or two triple bonds, (e) C₃₋₇cycloalkyl, (f —(CH₂)₀₋₂ (5 to 12 member) heteroaryl.

“R_{N-8}” is H, C₁₋₆alkyl, or phenyl.

“R_{N-10}” is H, C₁₋₆alkyl, C₃₋₇cycloalkyl, C₂₋₆alkenyl with one double bond, or C₂₋₆ alkynyl with one triple bond.

In a preferred embodiment of Formula (I): a=0, 1, 2, or 3 (a=0 or 1 being more preferred); b=0, 1, 2, or 3 (b=2 being more preferred); each R is independently halogen, OH, C₁₋₆alkyl, CN, C₁₋₆alkoxy, C₆₋₁₀aryl, (5 to 12 member) heteroaryl, wherein said alkyl and alkoxy may each optionally independently be substituted with up to three halogen (F preferred) or OH groups; (i.e. each and every R can be the same or different irrespective of the value of b). R* is H, C₁₋₆alkyl, —(CH₂)₀₋₅(C₆₋₁₀aryl), —(CH₂)₀₋₅ (5 to 12 member) heteroaryl, wherein said alkyl, aryl, or heteroaryl may each optionally independently be substituted with up to three halogen (F preferred), C₁₋₆alkoxy or OH groups; and Ar is selected from (i), (ii), (iii), or (iv) any of which Ar may be optionally substituted with a fluoro (F) at a ring carbon atom (preferably when Ar is (i)): 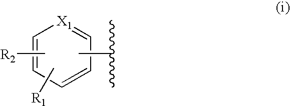
wherein:

X₁ is CH or N; R₁ is H, halogen (Br preferred), C₁₋₆alkyl, C₃₋₆cycloalkyl, C₂₋₁₂alkenyl; C₂₋₁₂alkynyl, (5 to 12 member) heteroaryl, OH, CN, SH, C₁₋₆alkoxy, S(C₁₋₆)alkyl, —NR₃(C═O)_{c}R₄, —NR₃SO₂R₄, —(CH₂)_{c}(C═O)R₅, —(CH₂)_{c}(C═O)OR₅, —(S═O)R₅, —S(═O)₂R₅ wherein c=0 or 1, R₃, R₄ and R₅ are each independently H, C₁₋₆alkyl, C₃₋₆cycloalkyl, C₂₋₆alkenyl, (CH₂)₀₋₅(C₆₋₁₀aryl), (CH₂)₀₋₅ (5 to 12 member) heteroaryl or NR₃(Y)R₄ wherein Y is CO or SO₂, and R₃ and R₄ together with the N and the C or S atoms of Y to which they are attached form a (5 to 7 member) heterocycloalkyl, and wherein any of said alkyl, cycloalkyl or heterocycloalkyl may be each be optionally independently substituted with up to three halogen (F preferred), OH, C₁₋₆alkyl, C₁₋₆alkoxy, or CN groups;

R₂ is independently —C(═O)R₃, —(C═O)_{c}NR₃R₄, —NR₃SO₂R₄ or —OR₅ wherein c=0 or 1, and R₃, R₄, and R₅ are as defined above, or R₂ is —NR₃SO₂R₄ wherein R₃ and R₄ together with the N and S atoms to which they are attached form a (5 to 7 member) heterocycloalkyl and wherein any of said alkyl, cycloalkyl or heterocycloalkyl moieties of R₂ may each be optionally independently substituted with up to three halogen (F preferred), OH, C₁₋₆alkyl, C₁₋₆ alkoxy or CN groups;

or R₁ and R₂ together with the C atoms to which they are attached form a fused C₅₋₁₀cycloalkyl, C₅₋₁₀aryl or (5 to 10 member) heteroaryl group wherein said fused cycloalkyl, aryl or heteroaryl group is optionally independently substituted with up to three groups selected from R₇ and R₈ wherein R₇ is C₁₋₆ alkyl said alkyl optionally substituted with up to three F, OH, C₁₋₃alkoxy groups; and R₈ is —(C═O)_{d}R₅ wherein d=0 or 1, and R₅is as defined above; 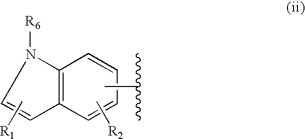
wherein:

R₁ and R₂ are as defined above in (i); and R₆ is H, C₁₋₆ alkyl, —(CH₂)₀₋₅(C₆₋₁₀aryl), —(CH₂)₀₋₅ (5 to 12 member) heteraryl, wherein said alkyl maybe optionally independently substituted with up to three halogen, C₁₋₆alkoxy or OH groups; 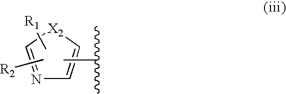
wherein:

X₂ is NH, N(C₁₋₆alkyl), O or S; and R₁ and R₂ are as defined above; or 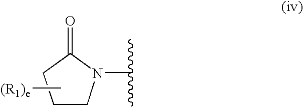
wherein:

e=1 or 2; and each R₁ is independently as defined above irrespective of the value of e (wherein when e=2, each R₁ is preferably —NH(C═O)_{c}(C₁₋₆alkyl), and C₁₋₆alkyl); preferably, when Ar is (iv), a is not zero; more preferably, when Ar is (iv), a=1.

In one preferred practice: Ar=(i); independently each R is halogen; a=0; b=2; and R₂ is —C(═O)_{c}NR₃R₄. More preferably, c=1; R₃ and R₄ are each C₃alkyl; R═F; and R₁ is C₁₋₆alkyl, halogen, a (5 to 12 member) heteroaryl or C₂₋₁₂alkynyl. Still more preferably, R₁ is methyl, bromine, oxazolyl or ethynyl.

In a second preferred practice: Ar=(ii); R₂═—C(═O)_{c}R₃; independently each R=halogen; a=0; and b=2. More preferably, c=1; R₃ and R₄ are each C₃alkyl; R═F; R₁═H; and R₆═C₁₋₆alkyl. Still more preferably, R₆═C₂-C₆alkyl. Representative compounds in this regard include:

In a particular practice the compound of the invention has Formula (Ia), whose constituents are as defined herein

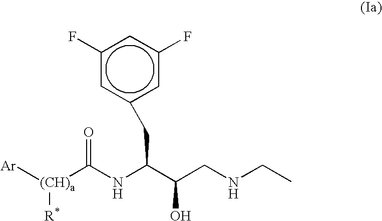

In a particularly preferred practice, the invention is of formula (Ib):

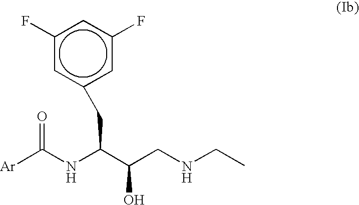

In particularly preferred practices, Ar is

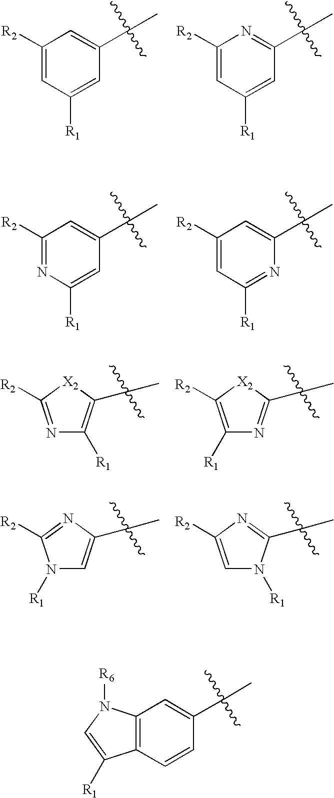

In more particularly preferred practices, Ar is:

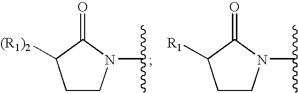

In another embodiment, the invention is to a pharmaceutical composition comprising the compound of Formula (I) and a pharmaceutically acceptable carrier, such carriers as known in the art.

In another preferred practice, the compound of Formulae (I) and (Ia) is as herein described with the proviso that it is not (1S,2R)N-[1(3,5-Difluoro-benzyl)-3-ethylamino-2-hydroxy-propyl)]-5-methyl-N′N′-dipropyl-isopthalamide.

In another embodiment, the invention is to a method of treating a CNS condition comprising administering to a patient in need of such treatment a therapeutically effective amount of the compound of Formula (I). Preferably, said CNS condition is a neurodegenerative condition, such as Alzheimer's Disease.

In another embodiment, the invention is to a method of treating a condition in which inhibition of beta-secretase is indicated comprising administering to a patient in need of such treatment a beta-secretase inhibiting amount of the compound of Formula (I).

CNS conditions subject of the invention are those known in the art; and include without limitation:

Head trauma, spinal cord injury, inflammatory diseases of the central nervous system, neurodegenerative disorders (acute and chronic), Alzheimer's Disease, demyelinating diseases of the nervous system, Huntington's disease, Parkinson's Disease, peripheral neuropathy, pin, cerebral amyloid angiopathy, nootropic or cognition enhancement, amyotrophic lateral sclerosis, multiple sclerosis, migraine, depression anorexia, Restless Leg Syndrome, dyskinesia associated with dopamine agonist therapy.

Anxiety or psychotic disorders such as: schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type. Examples of anxiety disorders include, but are not limited to, panic disorder; agoraphobia; a specific phobia; social phobia; obsessive-compulsive disorder; post-traumatic stress disorder; acute stress disorder; and generalized anxiety disorder.

Movement disorders involving: Huntington's disease and dyskinesia associated with dopamine agonist therapy; Parkinson's disease and restless leg syndrome.

Chemical dependencies: for example alcohol, amphetamine, cocaine, opiate, nicotine addiction.

Disorders comprising, as a symptom thereof, a deficiency in cognition: for example, a subnormal functioning in one or more cognitive aspects such as memory, intellect, or learning and logic ability, in a particular individual relative to other individuals within the same general age population. Also, any reduction in any particular individual's functioning in one or more cognitive aspects, for example as occurs in age-related cognitive decline. Examples of disorders that comprise as a symptom a deficiency in cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; and age-related cognitive decline.

Mood disorders or mood episodes such as: major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder.

In one embodiment, disorders subject to treatment by the invention include those selected from: hypertension, depression (e.g. depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g. agoraphobia, social phobia and simple phobias), posttraumatic stress syndrome, avoidant personality disorder, premature ejaculation, eating disorders (e.g. anorexia nervosa and bulimia nervosa), obesity, chemical dependencies (e.g. addictions to alcohol, cocaine, heroin, phenobarbital, nicotine and benzodiazepines), cluster headache, migraine, pain, Alzheimer's disease, obsessive-compulsive disorder, panic disorder, memory disorders (e.g. dementia, amnestic disorders, and age-related cognitive decline (ARCD), Parkinson's diseases (e.g. dementia in Parkinson's disease, neuroleptic-induced parkinsonism and tardive dyskinesias), endocrine disorders (e.g. hyperprolactinaemia), vasospasm (particularly in the cerebral vasculature), cerebellar ataxia, gastrointestinal tract disorders (involving changes in motility and secretion), negative symptoms of schizophrenia, schizoaffective disorder, obsessive compulsive disorder, mania, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania and headache (associated with vascular disorders).

Preferably, the CNS condition is a neurodegenerative condition. Representative neurodegenerative conditions preferably include without limitation those in which plaques comprised of beta-amyloid in whole or in part are associated, and/or in which the inhibition of beta-secretase is indicated. By way of example only, such conditions include Alzheimer's disease, Parkinson's Disease, Multiple Sclerosis, inclusion body myositis. In other embodiments, the invention pertains to treating a neurodegenerative condition comprising administering to a patient in need of such treatment a therapeutically effective amount of the instant compound; and to treating a condition in which the inhibition of beta-secretase is indicated by administering an inhibitory effective amount of said compound.

The compound of the invention can also be used in combination with other drugs, e.g. those conventionally used to treat any of the CNS conditions herein described. For example, the compound of the invention can be used in combination with any or all of the following to treat CNS conditions: neurodegenerative diseases such as Alzheimer's Disease: acetylcholinesterase inhibitors, such as donepezil, memantine, ACAT inhibitors, COX_{—}2 inhibitors, propentofyline, metryfonate, Vitamin E, Folic acid etc.; Parkinson's Disease: deprenyl, cabergoline, samanirole, L-dopa, mirapex, MAOB inhibitors such as selegine and rasagiline, comP inhibitors such as tasmar, A-2 inhibitors, dopamine reuptake inhibitors, NMDA antagonists, nicotime agonists, dopamine agonists and inhibitors of nitric oxide synthase (NOS), antidepressants such as selective serotonin reuptake inhibitors (SSRIs, sertraline).

Administration is by means known in the art. The compound can thus be administered alone or in combination with pharmaceutically acceptable carriers or other therapeutic agents, e.g. other neurodegenerative active agents, psychotropics etc. Dosage forms include without restriction: tablets, powders, liquid preparations, injectable solutions and the like.

The compound of the invention may be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration, the composition may take the form of tablets or lozenges formulated in conventional manner.

The compound of the invention may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogen-free water, before use.

The compound of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insulator may be formulated containing a powder mix of a compound of the invention and a suitable powder base such as lactose or starch.

A proposed dose of the compound of the invention for oral, parenteral or buccal administration to the average adult human for the treatment of the conditions referred to above is about 0.1 to about 200 mg of the active ingredient per unit dose which could be administered, for example, 1 to 4 times per day.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

In connection with the use of the compound of the invention it is to be noted that it may be administered either alone or in combination with pharmaceutically acceptable carriers by either of the routes previously indicated, and that such administration can be carried out in both single and multiple dosages. More particularly, the compound alone or in combination combination can be administered in a wide variety of different dosage-forms, i.e. they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 90% by weight of the total composition,

A proposed daily dose of the compound of the invention in the combination formulation (a formulation containing the compound of the invention and e.g. an acetlycholinase inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of Formula I per unit dose which could be administered, for example, 1 to 4 times per day.

Aerosol combination formulations for treatment of the conditions referred to above in the average adult human are preferably arranged so that each metered dose or “puff” of aerosol contains from about 0.01 mg to about 100 mg of the active compound of this invention, preferably from about 1 mg to about 10 mg of such compound. Administration may be several times daily, e.g. 2, 3, 4 or 8 times, giving for example, 1, 2 or 3 doses each time.

In practice, the IC50 of the compound of the invention in a BACE assay as described herein is about 600 nanomolar or less; preferably about 200 nanomolar or less, more preferably about 50 nanomolar or less.

Cell Free BACE1 Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds. The substrate is Biotin-GLTNIKTEEISEISYˆEVEFR—C [oregon green] KK—OH. The enzyme (0.1 nanomolar) and test compounds (0.00002-200 micromolar) are incubated in pre-blocked, low affinity, black plates (384 well) at RT for 30 minutes. The reaction, is initiated by addition of 150 millimolar substrate to a final volume of 30 microliter per well. The final assay conditions are: 0.00002-200 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin (0.75 micromolar). After incubation with streptavidin at room temperature for 15 minutes, fluorescence polarization is measured, for example, using a PerkinElmer Envision (Ex485 nm/Em530 nm). The activity of the beta-secretase enzyme is detected by changes in the fluorescence polarization that occur when the substrate is cleaved by the enzyme. Incubation in the presence of compound inhibitor demonstrates specific inhibition of beta-secretase enzymatic cleavage of its synthetic APP substrate.

In preferred practices, the N-methyl compound of the invention exhibits unexpectedly improved liver microsome stability.

The ensuing methods and examples illustrate, without limitation, representative ways to make the compound of the invention.

### Methods of Preparation

As used herein: Ac=acetyl; Boc=t-butoxycarbonyl; EDCI=1,(3,dimethylaminopropyl)-3-ethyl-carbiimide hydrochloride; CBZ=benzyloxycarbonyl; THF=tetrahydrofuran; DPPP=1,3-bis(diphenylphosphanyl)propane; dba=dibenzylideneacetone; Et=ethyl; Me=methyl; n-Bu=n-butyl; n-Hex=n-hexyl.

The compounds of this invention, 5, may be prepared by the sequence of reactions shown in Scheme 1. Epoxide 1 is reacted with an alkali metal-halide salt, preferably NaI, in the presence of a buffer, preferably HOAc/NaOAc, to give halohydrin 2. The reaction is performed between a temperature range of 0° C. to 60° C., preferably 25° C. The Boc-protecting group is removed by treatment with a strong acid, preferably aqueous HF, in a solvent such as acetonitrile, and the resulting amine salt is acylated with Ar[CHR**]ₐCO₂H using a coupling reagent well-known to one skilled in the art, preferably EDCI, in the presence of base, preferably a tertiary amine such as triethylamine, to give amide 3. Alternatively, Ar[CHR**]ₐCO₂H may be converted to the corresponding acid chloride using thionyl or oxalyl chloride and likewise reacted with the amine salt in the presence of a base. The reaction is performed between a temperature range of 0° C. to 60° C., preferably 25° C. Hydroxy amide 3 is protected as the dimethyl acetonide derivative 4 using 2-methoxypropene in the presence of an acid such as a sulfonic acid, preferably p-toluenesulfonic acid. The reaction is performed between a temperature range of 0° C. to 60° C., preferably 25° C. The halide group of 4 is displaced by ethylamine by heating with an excess of the amine in an inert solvent, preferably THF. The reaction is performed between a temperature range of 25° C. to 150° C., preferably 55° C. when the halide is iodide. The product is subjected to hydrolysis by heating in a mixture of a strong aqueous acid, preferably HCl, and an alcoholic solvent, preferably methanol, between a temperature range of 35° C. to 100° C., preferably 55° C., to give compounds 5.

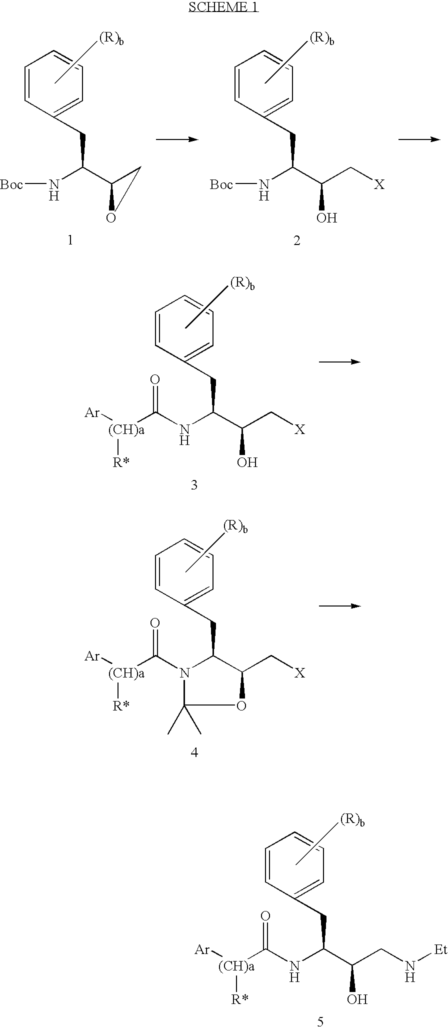

The compounds of this invention, 5, may also be prepared by the sequence of reactions shown in Scheme 2. Epoxide 6 is reacted with ethylamine in an alcoholic solvent, preferably isopropanol, between a temperature range of 0° C. to 50° C., preferably 25° C., to give amino alcohol 7. The NH group is protected as a t-butoxycarbonyl derivative by treatment with di-t-butyl-dicarbonate in the presence of a tertiary amine, preferably triethylaime, to give 8. The reaction is performed between a temperature range of 0° C. to 50° C., preferably 25° C. The CBZ group of 8 is removed to give amine 9 by catalytic hydrogenolysis in an inert solvent, preferably methanol, at a hydrogen pressure of 1 to 5 atmospheres and a temperature range of 0° C. to 50° C., preferably 25° C. The preferred catalyst is palladium but others well-known to one skilled in the art may be substituted. Amine 9 is acylated with Ar[CHR*]ₐCO₂H using a coupling reagent well-known to one, skilled in the art, preferably EDCI, in the presence of a base, preferably a tertiary amine such as triethylamine, to give amide 10. Alternatively, Ar[CHR*]ₐCO₂H may be converted to the corresponding acid chloride using thionyl or oxalyl chloride and likewise reacted with amine 9 in the presence of a base. The reaction is performed between a temperature range of 0° C. to 60° C., preferably 25° C. The Boc-protecting, group of 10 is removed by treatment with a strong acid, preferably aqueous HF or HCl, in solvents such as acetonitrile or dioxane, respectively, to give 5.

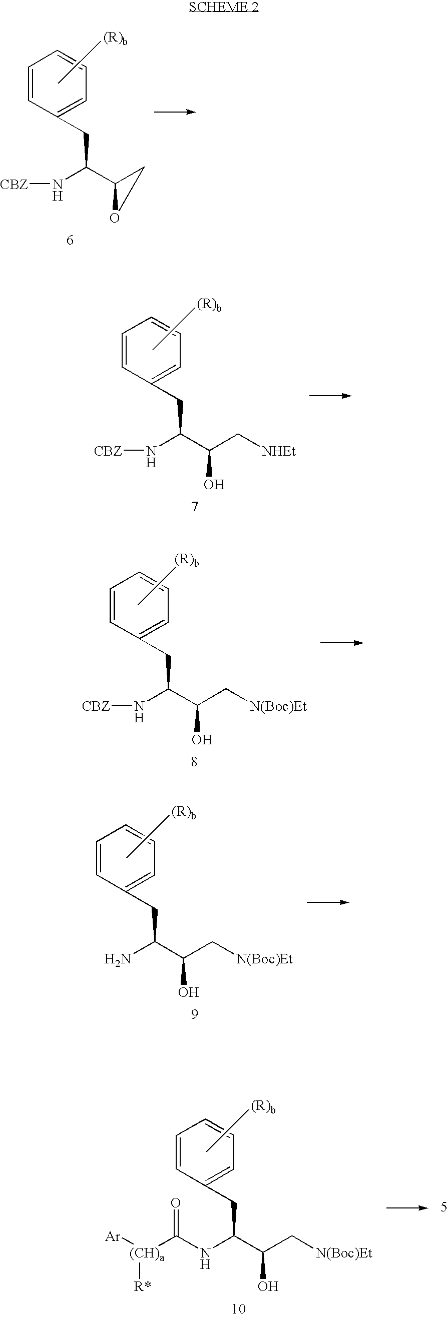

Intermediate 7 may also be prepared by the sequence of reactions shown in Scheme 3. Epoxide 1 is reacted with allylethyl amine in an alcoholic solvent, preferably isopropanol, between a temperature range of 0° C. to 50° C., preferably 25° C., to give amino alcohol 11. The Boc-protecting group of 11 is removed by treatment with a strong acid, preferably aqueous HF or HCl, in solvents such as acetonitrile or dioxane, respectively, to give 12. Protection of the NH₂ group of 12 is accomplished by treatment with benzyl chloroformate in the presence of a base, preferably pyridine or aqueous NaHCO₃ solution, and in an inert solvent, preferably CH₂Cl₂, THF or dioxane, between a temperature range of −15° C. to 50° C., preferably 0° C., to give 13. The allyl group of 13 is removed by treatment with N,N-dimethylbarbituric acid in the presence of a transition metal catalyst, preferably Pd₂(dba)₃/DPPP, in an inert solvent, preferably THF, between a temperature range of 25° C. to 100° C., preferably 60° C. to give 7.

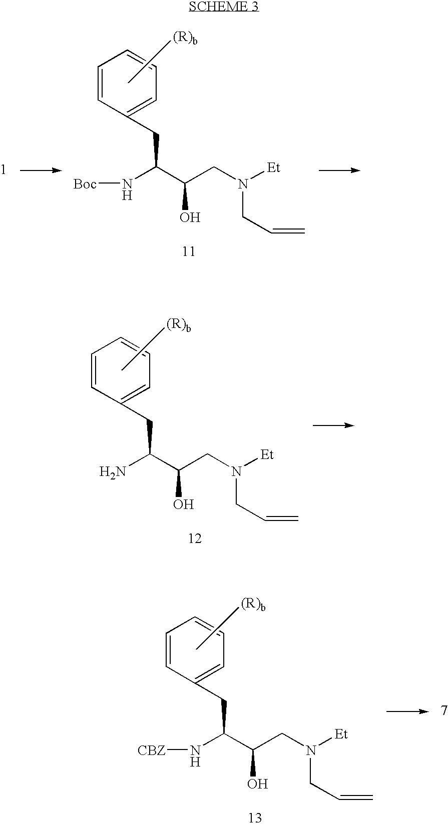

1. A compound of Formula I: 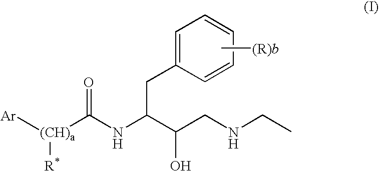
wherein:
a=0, 1, 2, or 3;
b=0, 1, 2, or 3;
each R is independently halogen, OH, CN, SH, NH₂, C₁₋₆alkyl, C₁₋₆alkoxy, S(C₁₋₆alkyl), NH(C₁₋₆alkyl), N(C₁₋₆alkyl)(C₁₋₆alkyl), NHC(═O)O(C₁₋₆alkyl), NHSO₂(C₁₋₆alkyl), C(═O)NH(C₁₋₆alkyl), C(═O)N(C₁₋₆alkyl)(C₁₋₆alkyl), C₆₋₁₀aryl, (5 to 12 member) heteroaryl, wherein each alkyl group aforesaid may be independently optionally substituted with up to three F, OH or C₁₋₃alkoxy groups;
R* is H, C₁₋₆alkyl, —(CH₂)₀₋₅(C₆-C₁₀aryl), —(CH₂)₀₋₅ (5 to 12 member) heteroaryl; and
Ar is selected from (A), (B), (C), (D), (E) or (F):
(A) C₆₋₁₀aryl, (5 to 12 member) heteroaryl, (C₆₋₁₀aryl)-W—(C₆₋₁₀aryl), (C₆₋₁₀aryl)-W-(5 to 12 member) heteroaryl, (C₆₋₁₀aryl)-W-(5 to 7 member) heterocycloalkyl, (5 to 12 member) heteroaryl-W—(C₆₋₁₀aryl), (5 to 12 member) heteroaryl-W-(5 to 12 member) heteroaryl, (5 to 12 member) heteroaryl-W-(5 to 7 member) heterocycloalkyl, (5 to 7 member) heterocycloalkyl-W—(C₆₋₁₀aryl), (5 to 7 member) heterocycloalkyl-W-(5 to 12 member) heteroaryl, (5 to 7 member) heterocycloalkyl-W-(5 to 7 member) heterocycloalkyl, wherein W is selected from —(CH₂)₀₋₄—, —O—, —C(═O)—, —S(═O)₀₋₂—, —N(R_{N-5})—;
(B) —C(═O)(C₁₋₁₀alkyl) where alkyl is optionally independently substituted with up to three substitutents (“SB”) selected from: OH; C₁₋₆alkoxy; C₁₋₆thioalkoxy; C(═O)OR_{N-8}; —C(═O)NR_{N-2}R_{N-3}; —C(═O)R_{N-4}; —SO₂(C₁₋₈alkyl); —SO₂NR_{N-2}R_{N-3}; —NHC(═O)(C₁₋₆alkyl); —NHC(═O)OR_{N-8}; —NR_{N-2}R_{N-3}; —R_{N-4}; —OC(═O)(C₁₋₆alkyl); —O—C(═O)NR_{N-8}R_{N-8} where each R_{N-8} is the same or different; —O(C₁₋₆alkyl)C(═O)OH; —O—(C₁₋₆alkyl optionally substituted with up to three halogens); —NHSO₂(C₁₋₆ alkyl); F; Cl;
(C) —C(═O)(C₁₋₆alkyl)O(C₁₋₆alkyl) where each alkyl is optionally independently substituted with up to three substituents SB as defined above in (A);
(D) —C(═O)(C₁₋₆alkyl)S(C₁₋₆alkyl) where each alkyl is optionally independently substituted with up to three of substituents SB as defined above in (A);
(E) —C(═O)CH(—(CH₂)₀₋₂—O—R_{N-10})—(CH₂)₀₋₂—(C₆₋₁₀aryl), or —C(═O)CH(—CH₂)₆₋₂—O—R_{N-10}—(CH₂)₆₋₂— (9 to 12 member) heteroary or (F) —C(═O)(C₃₋₈cycloalkyl) where said cycloalkyl is optionally independently substituted with up to two substituents selected from: —(CH₂ )₀₋₄OH; —(CH₂)₀₋₄C₁₋₆alkoxy; —(CH₂)₀₋₄C₁₋₆thioalkoxy; —(CH₂)₀₋₄C(═O)—O—R_{N-8}; —(CH₂)₀₋₄C(═O)—NR_{N-2}R_{N-3}; —(CH₂)₀₋₄C(═O)—R_{N-4}; —(CH₂)₀₋₄SO₂—(C₁₋₆alkyl); —(CH₂)₀₋₄SO₂—NR_{N-2}R_{N-3}; —(CH₂)₀₋₄NH—C(═O)—C₁₋₆alkyl); —NH—C(═O)—O—R_{N-8}; —(CH₂)₀₋₄NR_{N-2}R_{N-3}; —(CH₂)₀₋₄R_{N-4}; —O—C(═O)—(C₁₋₆alkyl); —O—C(═O)—NR_{N-8}R_{N-8} where each R_{N-8} is the same or different; —O—(C₁₋₆alkyl)—C(═O)OH; —O—(C₁₋₆alkyl, wherein said alkyl is optionally substituted with up to three halogens); —NHSO₂(C₁₋₆alkyl); F; Cl;
R_{N-2} and R_{N-3} are each independently selected from the group (a) H; (b) C₁₋₆alkyl optionally substituted with one substituent selected from: OH or NH₂; (c) C₁₋₆alkyl optionally substituted with up to three halogen; (d) C₃₋₇cycloalkyl; (e) -(C₁₋₂alkyl)(C₃₋₇cycloalkyl); (f) —(C₁₋₆alkyl)O(C₁₋₃alkyl); (g) C₂₋₆alkenyl with one or two double bonds; (h) C₂₋₆alkynyl with one or two triple bonds; (i) C₁₋₆alkyl chain with one double bond and one triple bond; (j) C₆₋₁₀aryl; or (k) (5 to 12 member) heteroaryl;
R_{N-4} is morpholinyl, thiomorpholinyl, piperazinyl, piperidinyl, homomorpholinyl, homothiomorpholinyl, homothiomorpholinyl S-oxide, homothiomorpholinyl S,S-dioxide, pyrrolinyl and pyrrolidinyl where each group is optionally substituted with one, two, three, or four of C₁₋₆alkyl;
R_{N-5} is (a) C₁₋₆alkyl, (b) —(CH₂)₀₋₂(C₆₋₁₀aryl), (c) C₂₋₆alkenyl containing one or two double bonds, (d) C₂₋₆alkynyl containing one or two triple bonds, (e) C₃₋₇cycloalkyl, (f) —(CH₂)₀₋₂ (5 to 12 member) heteroaryl; and
R_{N-8} is H, C₁₋₆alkyl, or phenyl;
with proviso that said compound is not (1S,2R)N-[1(3,5-Difluoro-benzyl)-3-ethylamino-2-hydroxy-propyl)]-5-methyl-N′N′-dipropyl-isophthalamide.

2. A compound of Formula I: 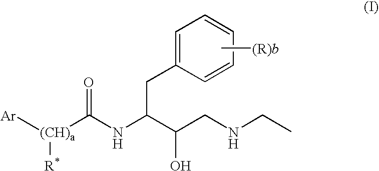
wherein:
a=0, 1, 2, or 3;
b=0, 1, 2, or 3;
each R is independently halogen, OH, C₁₋₆alkyl, CN, C₁₋₆alkoxy, C₆₋₁₀aryl, (5 to 12 member) heteroaryl, wherein said alkyl and alkoxy may each optionally independently be substituted with up to three halogen or OH groups;
R* is H, C₁₋₆alkyl, —(CH₂)₀₋₅(C₆₋₁₀aryl), —(CH₂)₀₋₅ (5 to 12 member) heteroaryl, wherein said alkyl, aryl or heteroaryl may each optionally independently be substituted with up to three halogen, C₁₋₆alkoxy or OH groups; and Ar is selected from (i), (ii), (iii) or (iv), any of which Ar may be optionally substituted with an F at a ring carbon atom: 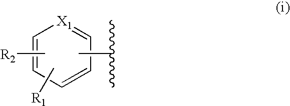
wherein:
X₁ is CH or N; R, is H, halogen (Br preferred), C₁₋₆alkyl, C₃₋₆cycloalkyl C₂₋₁₂alkenyl, C₂₋₁₂alkynyl, OH, CN, SH, C₁₋₆alkoxy, S(C₁₋₆)alkyl, —NR₃(C═O)_{C}R₄, —NR₃SO₂R₄, —(CH₂)_{C}(C═O)R₅, —(CH₂)C(C═O)OR₅, —(S═O)R₅, —S(═O)₂R₅ wherein c=0 or 1, R₃, R₄ and R₅ are each independently H, C₁₋₆alkyl, C₃₋₆cycloalkyl, C₂₋₆alkenyl or NR₃(Y)R₄ wherein Y is CO or SO₂ and R₃ and R₄ together with the N and the C or S atoms of Y to which they are attached form a (5 to 7 member) heterocycloalkyl, and wherein any of said alkyl, cycloalkyl or heterocycloalkyl may be each be optionally independently substituted with up to three halogen, OH, C₁₋₆alkyl, C₁₋₆alkoxy, or CN groups;
R₂ is independently —C(═O)R₃, —(C═O)_{c}NR₃R₄, —NR₃SO₂R₄ or —OR₅ wherein c=0 or 1, and R₃, R₄, and R₅ are as defined above, or R₂ is -NR₃SO₂R₄ wherein R₃ and R₄ together with the N and S atoms to which they are attached form a (5 to 7 member) heterocycloalkyl and wherein any of said alkyl, cycloalkyl or heterocycloalkyl moieties of R₂ may each be optionally independently substituted with up to three halogen, OH, C₁₋₆alkyl, C₁₋₆ alkoxy or CN groups;
or R₁ and R₂ together with the C atoms to which they are attached form a fused C₅₋₁₀cycloalkyl, C₅₋₁₀aryl, (5 to 10 member) heteroaryl group wherein said fused cycloalkyl, aryl or heteroaryl group is optionally independently substituted with up to three groups selected from R₇ and R₈ wherein R₇ is C_{1 6} alkyl said alkyl optionally substituted with up to three F, OH, Clalkoxy groups; and R₈ is —(C═O)_{d}R₅ wherein d=0 or 1, and R₅ is as defined above; 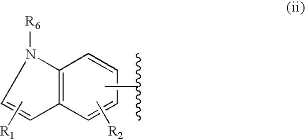
wherein:
R₁ and R₂ are as defined above in (i); and R₆ is H, C₁₋₆ alkyl, —(CH₂)₀₋₅(C₆₋₁₀aryl), —(CH₂)₀₋₅(5 to 12 member) heteroaryl, wherein said alkyl maybe optionally independently substituted with up to three halogen, C₁₋₆alkoxy or OH groups; 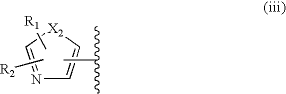
wherein:
X₂ is NH, N(C₁₋₆alkyl), O or S; and R₁ and R₂ are as defined above; or 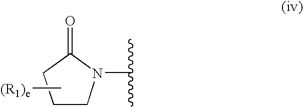
wherein:
e=1 or 2; and each R₁ is independently as defined above, and wherein when Ar is (iv), a=1;
with proviso that said compound is not (1S,2R)N-[1(3,5-Difluoro-benzyl)-3-ethylamino-2-hydroxy-propyl)]-5-methyl-N′N′-dipropyl-isophthalamide.

3. The compound of claim 2 wherein
a=0;
b=2;
each R is independently a halogen;
Ar is (i); and
R₂ is —(C═O)_{c}NR₃R₄.

4. The compound of claim 4 wherein
R is F;
c=1; and
R₃ and R₄ are each independently C₃alkyl;
R₁ is C₁₋₆alkyl, halogen, a (5 to 12 member) heteroaryl, or C₂₋₁₂alkynyl.

5. The compound of claim 4 wherein
R₁ is methyl, bromine, oxazolyl, or ethynyl.

6. The compound of claim 2 wherein
a=0;
b=2;
R₂ is —(C═O)_{c}NR₃R₄;
each R is independently a halogen; and
Ar is (ii).

7. The compound of claim 6 wherein
R is F;
c=1;
R₁ is H;
R₃ and R₄ are each C₃alkyl; and
R₆ is C₁₋₆alkyl.

8. The compound of claim 7 wherein
R₆ is C₂alkyl, C₄alkyl or C₆ alkyl.

9. A compound of Formula (Ib): 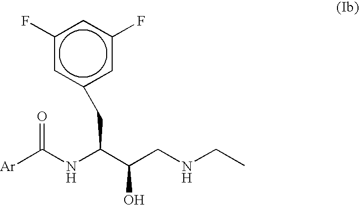
wherein:
Ar is selected from (i), (ii) or (iii): 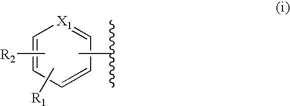
wherein:
X₁ is CH or N; R₁ is H, halogen, C₁₋₆alkyl C₃₋₆cycloalkyl, C₂₋₁₂alkenyl, C₂₋₁₂alkynyl, OH, CN, SH, C₁₋₆alkoxy, S(C₁₋₆)alkyl, —NR₃(C═O)R₄, —NR₃SO₂R₄, —(CH₂)_{C}(C═O)R₅, —(CH₂)C(C═O)OR₅, —(S═O)R₅, —S(═O)₂R₅ wherein c =0 or 1, R₃, R₄ and R₅ are each independently H, C₁₋₆alkyl, C₃₋₆ cycloalkyl, C₂₋₆alkenyl or NR₃SO₂R₄ wherein R₃ and R₄ together with the N and S atoms to which they are attached form a (5 to 7 member) heterocycloalkyl, and wherein any of said alkyl, cycloalkyl, or heterocycloalkyl may be each be optionally independently substituted with up to three halogen, OH, C₁₋₆alkyl, C₁₋₆alkoxy, or CN groups;
R₂ is independently —C(═O)R₃, —(C═O)_{c}NR₃R₄, —NR₃SO₂R₄ or —OR₅ wherein c=0 or 1, and R₃, R₄, and R₅ are as defined above, or R₂ is —NR₃SO₂R₄ wherein R₃ and R₄ together with the N and S atoms to which they are attached form a (5 to 7 member) heterocycloalkyl and wherein any of said alkyl, cycloalkyl or heterocycloalkyl moieties of R₂ may each be optionally independently substituted with up to three halogen, OH, C₁₋₆alkyl, C₁₋₆alkoxy or CN groups;
or R₁ and R₂ together with the C atoms to which they are attached form a fused C₅₋₁₀cycloalkyl, C₅₋₁₀aryl, or (5 to 10 member) heteroaryl group wherein said fused cycloalkyl, aryl or heteroaryl group is optionally independently substituted with up to three groups selected from R₇ and R₈ wherein R₇ is C₁₋₆ alkyl said alkyl optionally substituted with up to three F, OH, C₁₋₃alkoxy groups; and R₈ is —(C═O)_{d}R₅ wherein d=0 or 1, and R₅ is as defined above; 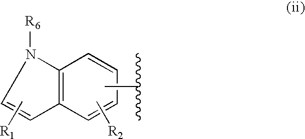
wherein:
R₁ and R₂ are as defined above in (i); and R₆ is H, C₁₋₆ alkyl, —(CH₂)₀₋₅(C₆₋₁₀aryl), —(CH₂)₀₋₅(5 to 12 member) heteroaryl, wherein said alkyl maybe optionally independently substituted with up to three halogen, C₁₋₆alkoxy or OH groups; 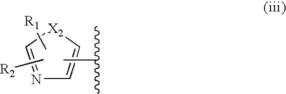
wherein:
X₂ is NH, N(C₁₋₆alkyl), O or S; and R₁ and R₂ are as defined above;
with the proviso that said compound is not (1S,2R)N-[1(3,5-Difluoro-benzyl)-3-ethylamino-2-hydroxy-propyl)]-5-methyl-N′N′-dipropyl-isophthalamide.

10. A pharmaceutical composition comprising the compound of claim 1, 2 or 9 and a pharmaceutically acceptable carrier.

11. A method of treating a CNS condition comprising administering to a patient in need of such treatment a therapeutically effective amount of the compound of claim 1.

12. The method of claim 11 wherein said CNS condition is a neurodegenerative condition.

13. The method of claim 12 wherein said neurodegenerative condition is Alzheimer's Disease.

14. A method of treating a condition in which inhibition of beta-secretase is indicated comprising administering to a patient in need of such treatment a beta-secretase inhibiting amount of the compound of claim 1, 2 or 9.
